(19) United States
(12) Patent Application Publication
(10) Pub. No.: US 2010/0278847 A1
(43) Pub. Date: Nov. 4, 2010

(54) MUTUALLY SUPPRESSIVE GENE/INHIBITOR COMBINATIONS FOR NON-ANTIBIOTIC SELECTION OF RECOMBINANT STRAINS

(76) Inventors: Liam Good, London (GB); Shan Goh, Hertfordshire (GB)

Correspondence Address: MORSE, BARNES-BROWN & PENDLETON, P.C.;ATTN: IP MANAGER, RESERVOIR PLACE, 1601 TRAPELO ROAD, SUITE 205, WALTHAM, MA, 02451, US

(21) Appl. No.: 12/669,156

(22) PCT Filed: Jul. 10, 2008
(86) PCT No.: PCT/SE2008/050864

§ 371 Date: Jun. 21, 2010

(30) Foreign Application Priority Data
Jul. 16, 2007 (SE) 0701739-5

(51) Int. Cl. A61K 39/00; C12N 15/63; C12N 5/10; C12P 1/00; C12P 1/04; C12P 1/02; C12Q 1/68; A61P 37/04

(52) U.S. Cl. 424/184.1; 435/320.1; 435/252.3; 435/254.11; 435/419; 435/41; 435/170; 435/171; 435/6

(57) ABSTRACT

This invention relates to vector sequences that express growth inhibitory levels of essential genes used in combination with inhibitors that target the same gene. An example given is the use of the gene encoding the fatty acid biosynthesis enzyme enoyl-ACP reductase used in combination with the antimicrobial triclosan. The expression level of the enoyl-ACP reductase is sufficient to suppress the toxic effects of triclosan and the triclosan is used at levels that are sufficient to suppress the toxic effects of the enoyl-ACP reductase. The invention provides methods to enhance the growth and survival of genetically modified organisms and to increase the production and expression of plasmid vectors in the presence of triclosan, relative to methods that rely on antibiotics and antibiotic resistance markers. Also, the invention provides methods to limit the spread of genetically modified organisms. The vectors and methods are useful to avoid the use of antibiotics and antibiotic markers, to contain genetically modified organisms and to increase the production of recombinant material or metabolites from host organisms.

### FIELD OF INVENTION

The present invention is in the field of molecular biology. More specifically, this invention pertains to nucleic acid fragments that are borne by plasmid vectors, which are commonly used to generate genetically modified organisms. The invention involves methods to enhance the growth and survival of the host organism and the selection, production and expression of the plasmid vector. The conditions required for improved growth and plasmid vector production and selection do not require antibiotics or antibiotic resistance genes, which are widely believed to pose risk to the environment, agriculture, medicine and health.

### BACKGROUND OF THE INVENTION

An essential requirement in genetic engineering of microorganisms is the capacity to select strains with defined genotypic alterations. Antibiotic resistance marker genes are commonly used in research and industry to select and maintain recombinant organisms in the presence of antibiotics. Unfortunately, such applications are likely to increase the problem of antibiotic resistance in medicine and agriculture and their use also posses environmental concerns (Paterson 2006). In addition, a common practical problem associated with antibiotic selection systems is poor plasmid stability, where plasmid-free cells arise due to enzymatic depletion of the antibiotic in the media during growth. Finally, there are regulatory constraints on the use of antibiotic resistance markers, particularly where biohazard strains or biopharmaceuticals are involved. For example, the introduction of antibiotic resistance markers into category A & B biohazard strains is discouraged or even prohibited. Also, DNA or recombinant vaccines are typically plasmid-based and must lack antibiotic resistance markers. Accordingly, there have been calls for alternative selective marker systems, but a viable alternative should still be convenient, robust and flexible. In the U.S., federal regulations specifically discourage the use of markers that confer resistance to antibiotics in significant clinical use in order to avoid unnecessary risks of spreading antibiotic resistance traits to environmental microbes. In addition to ensuring regulatory approval, removal of resistance markers increases the variety of plasmids that can be propagated within a single cell and removes the risk of altered expression of chromosomal genes adjacent to the antibiotic-resistance marker.

Antibiotics and antimicrobials are used liberally in many areas of medicine, agriculture, research and industry, and this contributes to the increase in the prevalence of resistant strains. In the US, the annual use of antimicrobials is 23 million kg (EU Commission, Scientific Health Opinions, 1999) and the cost attributed to resistant strain problems in medicine is greater than USD 30 billion per year (http://www.niaid.nih.gov/factsheets/antimicro.htm). Clearly, the situation calls for restraint and the development of alternative strategies.

There has been extensive development and application of antibiotic resistance markers used in combination with antibiotics to select and maintain genetically modified organisms. Within the field of microbiology, detailed methods are widely available.

In response to the need for reduced antimicrobial usage, there have been several attempts to develop antibiotic-free strain selection strategies for genetic engineering. A variety of strategies for antibiotic-free selection have been developed for plant and microbial systems. Unfortunately, the bacterial systems suffer from several limitations, which include requirements for particular host strain genetic backgrounds, defined media or expensive selective agents. The first non-antibiotic system, and still the most popular alternative, involves an auxotrophic bacterial strain and complementation using a plasmid-encoded biosynthesis gene, such that only transformants can grow on defined media lacking the nutrient (González et al. 1985). For example, the host may be deficient in the production of an essential amino acid or thermo-sensitive for growth, and selection involves growth under the selective condition. In other approaches, the host strain may carry lethal genes or genes conferring a metabolic burden under the control of a repressor that is used for selection (Williams et al. 1998). Although successful for strain selection, there are severe limitations with these systems. One common problem, which also applies to antibiotic selection, is that the selective component can leak to neighboring cells and compromise overall selection. Another problem can be the metabolic burden of the mutant phenotype or the need for special selective conditions that slow growth. An improved approach involves an additional plasmid sequence that binds repressor proteins that would otherwise sequester an essential gene (Cranenburgh et al. 2001). This allows the transformants to grow without added inductive or selective agents, but the method is limited to certain strains that are difficult to construct.

It has been shown that essential gene over-expression can raise inhibitor tolerance (Tennhammar-Ekman et al. 1986; (Stokes et al. 2005; (Xu et al. 2006). For example, it is known that the over-expression of fabI provides limited resistance to triclosan (Xu 2006) and that reduced expression of fabI sensitizes cells to triclosan (Dryselius et al. Good 2005). The use of the growth essential gene fabI and inhibitors including triclosan has been described for plasmid selection by (Stuetj et al. 2000). Also, (Li and Rendina 2000) have described methods to achieve altered levels of enoyl reductase in transformed cells that carry copies of the enoyl-ACP reductase gene. However, these publications and patents do not speak to the toxic effects of fabI expression, containment of a fabI marker in the absence of triclosan or strategies to combine fabI expression with triclosan treatment to achieve improved growth, plasmid stability, yield and expression relative to other plasmid selection systems

It has been shown that over-expression of essential genes that are targets of specific inhibitors results in inhibitor resistance (Xu et al. 2006). An E. coli essential gene that has been widely studied and which has displayed the above phenomenon is fabI (Heath, Yu, Shapiro, et al. 1998), which encodes for enoyl ACP reductase. It catalyses the elongation step of fatty acid biosynthesis, which is an important component of bacterial growth (Bergler et al. 1994). FabI is inhibited by triclosan, which binds specifically to the enoyl substrate site, inactivating the enzyme (Heath et al. 1998). Fatty acid biosynthesis is an essential growth process in most organisms. There are considered to be two diverged types of biosynthesis pathways, where the type II pathway is conserved in bacteria, fungi and plants. FabI (enoyl ACP reductase) catalyzes fatty acid elongation (Bergler et al. 1994). Interactions between triclosan, FabI and the NAD+ cofactor contribute to the formation of a stable FabI/NAD+/triclosan ternary complex, with the drug binding at the ACP-enoyl substrate site (Heath et al. 1999). Enoyl ACP reductase is a component of type II fatty acid biosynthesis pathway, which is conserved in microbes and absent in animals.

Triclosan is a polychloro phenoxy phenol of synthetic origin that was found to act as a broad-spectrum antimicrobial and now provides an inexpensive, stable and easy to handle additive. Although not used as a systemically administered drug, triclosan is commonly used in toothpaste (Panagakos et al. 2005), dental resins (Sehgal et al. 2007), topical medication for skin infections (Wohlrab et al. 2007), and even in plastics and textiles. Regulatory agencies have approved the use of triclosan in many applications. Triclosan has poor solubility in water, is stable at high temperatures for prolonged periods and is inexpensive.

Antibiotic dependent bacteria strains have been described for bacteria isolated from laboratories (Bertii and Caffau 1963; Kerasheva 1966; Dabbs and Looman 1981; (Goldstein et al. 2007) and clinics (Winstanley and Spencer 1987; Fraimow et al. 1994). However, there is no information available about the mechanism of dependence. In particular there is no indication that plasmid DNA or growth essential gene over-expression is involved in the dependence phenotype.

It is apparent from the discussion above that there is demand for new strategies based on growth essential genes and their inhibitor in the field of molecular biology and microbial production without employing antibiotics.

### SUMMARY OF INVENTION

In a general embodiment, the invention provides plasmid vectors that express inhibitory levels of a growth essential gene, or in other terms it expresses the growth essential gene in a host organism at a level that makes growth of host organism dependent on an inhibitor of the expressed gene. Preferably, the growth essential gene is the gene of enoyl-ACP reductase (fabI) or its homologues, where vector-mediated expression of the said gene reduces the growth or survival of a host organism by being capable of expressing toxic levels of fabI Also preferably, the vector has the open reading frame of the fabI gene is fused to an endogenous or heterologous gene sequence. The plasmid vectors preferably are pUC-derived plasmids and more preferably pUC-derived plasmids having bla replaced with the fabI gene.

In the context of the present invention, the term “a growth essential gene” is considered to be a gene that is needed in at least one copy in an organism's genome for the organism to grow under normal growth conditions. For example, the genes that are involved in core metabolic and biosynthetic processes such translation, transcription, cell wall biosynthesis are typically essential genes. In some cases essential genes have been identified as essential for growth, although the function of the gene is unknown or unclear. The term “express inhibitory levels of a growth essential gene” in the meaning of the present invention is such genes which are needed for growth, but with high levels of expression they become growth inhibitory or toxic. The persons skilled in this art are aware of hundreds of growth essential genes, and the list of growth essential genes in various organisms can be expected to increase along with better understand of their role in cells.

In another embodiment, the invention relates to a host organism transformed with the just described plasmid vectors. Suitable hosts are selected among, bacteria, fungi or plants.

In a further embodiment the invention relates to a method of enhancing growth or survival of the mentioned organisms by cultivation so the organisms expresses from the plasmid a toxic level of an essential gene in the presence of an inhibiting amount of an antibacterial compound that targets the over-expressed essential gene product, whereby the toxic effects from the plasmid vector are suppressed by said inhibitor and the toxic effects of the inhibitor are suppressed by said plasmid vector in a mutually suppressive combination. Preferably, the growth essential gene is the fabI gene and preferably the inhibitor is triclosan or a functional analogue of triclosan. Triclosan suitably is present in doses ranging from 100 nM to 10 μM, preferably 0.5 to 2 μM, and more preferably in an amount about 1 μM during the cultivation. The recited methods provides a number of specific advantages or improvements, besides increasing cell growth rates or yield of bacteria in a fermentation culture, such as increasing recombinant protein expression, increasing plasmid yield, increasing metabolite production, increasing bio-remediation activity of microorganisms, enhancing vector stability, improving cell lysis efficiency and limiting the dissemination or spread of plasmid vector sequences and thereby or limit the spread of genetically altered sequences or genetically modified organisms.

In an important aspect the invention relates to a method of producing plasmids while obtaining improvements in plasmid copy number, yield and stability. The method comprises the steps of transforming an organism with a plasmid vector capable of expressing the gene of enoyl-ACP reductase (fabI); culturing the organism under conditions of mutual suppression generated by the capacity of expressing toxic levels of enoyl-ACP reductase and the presence of a toxic, inhibitory amount of triclosan or a functional analogue of triclosan; and collecting the plasmids for further processing. Triclosan is present in an amount of 100 nM to 10 μM, preferably 0.5 to 2 μM, and more preferably in an amount about 1 μM. The plasmids may be further processed by inserting a heterologous or endogenous gene sequence with conventional methods, for example by fusion to fabI gene. The steps of transformation, collection and isolation included in the plasmid production method can be made with standard methodologies. Preferably, the organism is E. Coli and a number of suitable strains and handling procedures and are applicable with the method.

The plasmids obtained with described method can subsequently be used with production under the mentioned conditions. Also the invention provides methods, by fermentation, small molecule inhibitors capable of inhibiting growth essential genes using microorganisms that carry a vector according to what has been outlined above. The method comprises the steps of culturing such organisms in a manner that they produce as a metabolite a molecule that suppresses the toxic effects of the over-expressed gene.

Further, the invention encompasses a method of identifying or screening for small molecule inhibitors that bind and inhibit growth essential genes by culturing organisms that carry a vector according to what has been earlier disclosed, comprising the steps of subjecting selected inhibitor candidates to cultures and identifying molecules that are capable of suppressing the toxic effects of the growth essential gene.

The present invention also relates to a plasmid DNA vaccine product lacking an antibiotic resistance marker and a recombinant whole cell vaccine product lacking an antibiotic resistance marker produced according to the previously described methods.

By way of referring to a preferred example of the invention, the expression level of the enoyl-ACP reductase is sufficient to suppress the toxic effects of triclosan and the triclosan is used at levels that are sufficient to suppress the toxic effects of the enoyl-ACP reductase. This is referred to here as “mutual suppression”. Toxic levels of expression of the marker gene and toxic levels of triclosan when used in combination provide favorable and improved conditions for host growth and plasmid vector production and expression.

Other further aspects, features and advantages of the present invention will be apparent from the following detailed descriptions and figures.

### DETAILED AND EXEMPLIFYING DESCRIPTION

The present invention in relates one important example to an improved antibiotic-free cloning vector that uses the synthetic household biocide triclosan as a selective agent. Whereas conventional antibiotic resistance systems destroy or remove antibiotics by enzymatic action the fabI marker provides a protein target that sequesters triclosan in a stable complex (Ward et al. 1999), hence providing stable selection.

pFab is derived from pUC19 and preserves the origin of replication that defines its host range and all but three unique restriction sites in the MCS. The fabI cassette in pFab can be transferred easily into most pUC-derived plasmids. Therefore, pFab is compatible with the most popular cloning host strains and recombinant strategies. In addition, relative to its parent, pFab has an increased copy number, plasmid yield, and growth rate.

An unexpected interaction between fabI and triclosan was surprisingly observed, in this context termed reciprocal or mutual suppression. In general terms, reciprocal suppression is used to describe the neutralizing interaction between two otherwise negative effects. In the present case, one negative effect is the overexpression of fabI and the other negative effect is the presence of triclosan. The levels of overexpression and triclosan are such they that they independently reduce growth rates, as measured by monitoring culture optical densities, but in combination the negative effects are canceled and growth returns to normal or near normal rates. In short, fabI over-expression suppresses triclosan toxicity, and triclosan suppresses the toxic effects of fabI over-expression. This explains pFab instability in the absence of selection (Table 1) and in mixed culture with plasmid-free cells (FIG. 5). Cell toxicity mediated by over-expression of fabI could be due to de-regulation of the tightly coordinated fatty acid biosynthesis pathway. In the presence of triclosan, pFab cultures also displayed signs of stress, but grew faster during exponential phase and were more viable, suggesting triclosan suppressed dysfunction in fatty acid synthesis. Remarkably, addition of triclosan enabled pFab transformants to grow faster than pUC19 transformants. Therefore, we observed an unexpected interaction between fabI and triclosan which we call reciprocal suppression and this provides unexpected advantages with the pFab system. Therefore, there are unexpected advantages with the pFab system, which appear to arise through reciprocal suppression.

Reciprocal or mutual suppression has been described between two genes (Weitao, Nordström, and Dasgupta 1999) and inhibitors (Yue et al. 1963) and the present data reveals an example of gene/inhibitor reciprocal suppression. To our knowledge, there is no previous report of gene/inhibitor reciprocal suppression.

The combination of pFab type plasmid vectors and triclosan provides a new non-antibiotic selection marker system with many possible applications. Given that fabI is conserved in diverse bacteria, such as Bacillus subtilis (FabL) and Streptococcus pneumoniae (FabK), this system can likely be extended to other organisms that use type II fatty acid biosynthesis. In principle, the interaction could operate between any essential gene/inhibitor combinations. Also, pFab is attractive for large-scale production of recombinant proteins as it can be used to increase plasmid copy number, yield, and stability, and triclosan is inexpensive and easy to handle. Most importantly, the study shows that essential genes can be used in combination with non-antibiotic inhibitors to select and maintain recombinant bacteria, and reciprocal suppression provides a new paradigm to enhance the productivity of genetically modified organisms and to limit their spread.

The improved performance of the selectable marker system may reflect simple titration of excess toxic levels of FabI together with titration of toxic levels of triclosan to establish a physiological state that favors high levels of cell growth and plasmid vector production. However, this view may be overly simplistic or wrong and we make no claims regarding the exact mechanism(s) that lie behind the invention.

The expression level of the enoyl-ACP reductase is sufficient to suppress the toxic effects of triclosan and the triclosan is used at levels that are sufficient to suppress the toxic effects of the enoyl-ACP reductase. This is referred to here as “mutual suppression”, and could also be termed “reciprocal suppression”. Regardless of the terminology, the important point is that toxic levels of expression of the marker gene and toxic levels of triclosan when used in an appropriate combination provide favorable and improved host growth and plasmid vector production and expression.

In following exemplifying part, experiments illustrating the invention are given.

### BRIEF DESCRIPTION OF TABLE AND DRAWINGS

FIG. 1. Vector constructs. The MCS in pFab is similar to pUC19, except for HincII, HindIII and PstI, which are not unique in pFab. The fabI cassette in pFab can be transferred to other pUC-derived plasmids using the AatII and AlwNI restriction sites. The pBFab1 construct was derived from pBAD18s; pBFab6 derived from pBAD18 is not shown.

FIG. 2. Triclosan selection and resistance mediated by fabI over-expression. (a) Growth of pFab and pUCFA clones on LBT and LBA. (b) Triclosan resistance by arabinose-induced fabI expression. Growth rates were calculated as change in OD₅₅₀ over time during exponential growth. Growth rates of DH5α/pBFab1 and DH5α/pBFab6 relative to DH5α/pBAD18s and DH5α/pBAD18, respectively, are shown.

FIG. 3. Plasmid quality and abundance. (a) Plasmids from different E. coli hosts were digested with BamHI. (b) Ratio of pDNA to gDNA as a measure of copy number in pUC19 and pFab. Agarose gel electrophoresis of total DNA isolated from five clones of pUC19 and pFab. Bands were quantified by using ImageQuant software. (c) Mean ratio of pDNA:gDNA of pUC19 and pFab from (b).

FIG. 4. Effect of triclosan of the viability and fitness of pFab transformants. (a) Growth curves of DH5α/pUC19 and DH5α/pFab with and without selection. (b) Flow cytometric determination of dead cells, as a measure of cell toxicity, in DH5α/pFab cultures grown in various concentrations of triclosan. (c) Reduced fitness with fabI over-expression and its suppression by triclosan. Growth rates of pFab and pUCFA clones in LB and LBA, respectively, were expressed relative to DH5α/pUC19 in LBA.

FIG. 5. Growth competition between DH5α and DH5α/pUC19 or DH5α/pFab. The log 10 ratio of plasmid-bearing cells to total number of cells against time represents the rate of plasmid loss in mixed cell populations. The data is representative of two independent experiments.

FIG. 6. Schematic illustration of reciprocal suppression. Under normal physiological conditions, fabI expression provides FabI levels that are essential for growth (thin, grey, angled arrow), but when fabI is overexpressed the excess FabI produced is growth inhibitory (thick, forward angled, black line). The FabI inhibitor triclosan is toxic to cells (thick, back angled, black line). When toxic levels of FabI are present in combination with toxic levels of triclosan their effects may cancel each other out, providing conditions for reciprocal suppression (black circle). Such reciprocal suppression may increase cell growth and the stability and production of plasmids that carry fabI (thin, grey, vertical arrow).

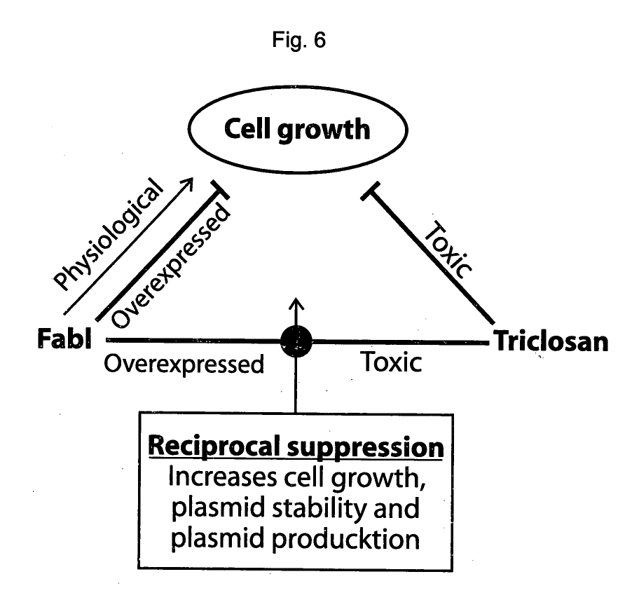

### METHODOLOGY AND EXPERIMENTS

### Bacterial Strains, Plasmids and Media

The E. coli strains used in this study were DH5α (Invitrogen), XL1-Blue (Strategene), HB101, BL21 and K12. Plasmids used in this study were pUC19 (New England Biolabs), pBAD18 and pBAD18s (National Institute of Genetics, Japan). Media were SOC and LB (GIBCOBRL) supplemented with ampicillin (LBA, 100 μg/ml ampicillin, (Sigma)), triclosan (LBT, 1 μM triclosan (Ciba) prepared from a 1 M stock in DMSO (Sigma)) and arabinose (Sigma).

Construction of pFab, pUCFA and pBFab

An EagI site was created at nt. 1621 of pUC19 (New England Biolabs), immediately downstream of bla, by PCR with primers (5′ cgtcggccgttaccaatgcttaatcag and 5′ cgccggccggaccaagtttactcatat). The amplicon was digested with EagI (New England Biolabs), ligated with T4 DNA ligase (Fermentas) and transformed into DH5α (Invitrogen) for propagation. The bla gene was excised from pUC19 with SspI (New England Biolabs) and EagI, and replaced with fabI together with its promoter, which was amplified from K12 genomic DNA with primers (5′ ccggatatcgtgctggagaatattcg and 5′ gcgcggccgttatttcagttcgagttcgtt) and then digested with EcoRV (New England Biolabs) and EagI to create the pFab vector. The pFab vector was transformed into DH5α and plated onto LB with 0.5-5 μM triclosan. Transformants were subsequently maintained in 1 μM triclosan (LBT).

The fabI gene was also cloned into pUC19 at SphI (New England Biolabs) and BamHI (New England Biolabs) within the MCS (pUCFA). Primers (5′ ccggcatgcgtgctggagaatattcg and 5′ ccggatccgattatttcagttcgagt) were used for amplification of fabI in K12. The pUCFA vector was transformed to DH5α and plated onto LBA and LBT.

To induce expression of fabI from P_{BAD}, the fabI amplicon generated from primers (5′ cggaattcgaatgggttttctttccgg and 5′ cctctagagattatttcagttcgagt) was digested with EcoRI and XbaI (New England Biolabs) and cloned into pBAD18s, which was similarly digested, to yield pBFab1. Expression of fabI in pBAD18 required a Shine Dalgrano sequence, which was predicted to be uaagga at position −13 relative to the start codon. Primers (5′ cggaattctcaacaataaggattaaagc and 5′ cctctagagattatttcagttcgagt) were used for amplification of fabI with its Shine Dalgarno sequence, and cloned into pBAD18 to yield pBFab6. DH5α were transformed with the pBFab plasmids and plated onto LBA, LBT and LBT with 0.2% (w/v) arabinose.

### Plasmid and Strain Properties

Transformation efficiency of plasmids by heat-shock of chemically competent DH5α cells was determined as recommended by manufacturer (Invitrogen).

Plasmid yields from one ml overnight cultures grown under selection were determined from five clones of pUC19 and pFab. Plasmids were isolated using a miniprep kit (Qiagen) and quantified by OD₂₆₀ readings. Plasmids (50 ng) were digested with BamHI and electrophoresed in 1% agarose gel.

Plasmid stability with selection was determined as a percentage of blue colonies to total colonies formed on selective plates with X-Gal (20 μg/ml, Saveen), from a 16 h culture grown in LBA or LBT. Plasmid stability without selection was determined as the percentage of blue colonies to total colonies on LB plates with X-Gal (20 μg/ml), from a 16 h culture grown in LB. Five clones of pUC19 and pFab were used.

Plasmid abundance was determined in two ways. First, to compare band intensities of genomic (gDNA) to plasmid DNA (pDNA) in an agarose gel, total genomic DNA was extracted from five different clones of pUC19 and pFab clones grown under selection for 16 h. Five cultures of K12, derived from five single colonies, were grown in LB for DNA extraction using the Bacterial GenElute system (Sigma) for one ml of overnight culture. Total DNA (10 μl) was electrophoresed in a 1% agarose gel, which was stained with ethidium bromide and scanned by Typhoon 9400 (Amersham Biosciences). Band intensities of pDNA to gDNA were determined by ImageQuant (Amersham Biosciences).

Second, relative quantitative PCR (qPCR) was carried out by using the plasmid lacZα gene as the target gene and single copy chromosomal dxs as the reference gene (Lee et al. 2006). K12 gDNA containing a single copy of lacZα and dxs was used as a calibrator. Primers amplifying the target gene (5′ gtgctgcaaggcgattaagtt and 5′ cactggccgtcgttttacaa), and reference gene (5′ cgagaaactggcgatcctta and 5′ cttcatcaagcggtttcaca) were validated for similar amplification efficiencies. Hence, real time data analyses were carried out by the 2^{-ΔΔCT} method for relative qPCR (Livak and Schmittgen 2001). Total DNA concentrations were determined by OD₂₆₀ absorbance for qPCR. Each 25 μl of PCR reaction contained 12.5 μl of SYBR Green PCR buffer (Eurogentec), 100 nM of each primer (Biomers) and 5 ng total DNA.

Growth rate was calculated from the exponential phase of growth, which was monitored as increased OD₅₅₀ over time by the VERSAmax spectrophotometer (Molecular Devices). An overnight culture (16 h), standardized by OD550 to yield approximately 7×10⁵ cfu/ml, was grown in 200 μl volumes per well in a 96-well plate for 24 h with agitation for 5 s every 5 min, when OD₅₅₀ readings were taken. Triclosan dissolved in DMSO was added to give 0-2 μM Triclosan and 1% DMSO final concentrations.

The host range of pFab within commonly used E. coli cloning strains was tested by transformation of XL1-Blue (Stratagene), HB101 and BL21, selection on LBT, plasmid extraction and digestion of 100 ng DNA with BamHI. DNA integrity was assessed in a 1% agarose gel.

Inducible Expression of fabI

Clones of pBFab1 and pBFab6 were grown in LBA for 16 h, diluted to approximately 5-9×10⁶ cfu/ml in LBAT and aliquoted into 180 μl volumes per well in a 96 well plate. Arabinose was added to a final concentration of 0-5% and the final volume per well was made up to 200 μl with water. Clones of pBAD18 and pBAD18s were included as controls. Cultures were grown for 24 h in the VERSAmax spectrophotometer (Molecular Devices) with agitation for 5 s every 5 min, when OD₅₅₀ readings were taken. The growth rate at each arabinose concentration was calculated as described above.

### Cell Viability

DH5α/pFab were grown overnight in the absence or presence of 0.5-2 μM triclosan and subjected to SYTOX green staining flow cytometry, as previously described (Roth et al. 1997). DH5α/pUC19 was included as a control for green fluorescence of live and heat-treated dead cells. Samples were excited with a 488 nm air-cooled argon ion laser in the CyFlow SL flow cytometer (Partec GmbH). Threshold settings were enabled on forward scatter to exclude cell debris. The forward and side scatter dot plot was used to identify and gate cell populations. Fluorescence was measured at 520 nm Viable and dead cell populations were counted using the Partec FloMax software version 2.4e.

### Competition Experiment

Growth competition between DH5α and plasmid bearing cells was carried out as previously described, with modifications (Lenski et al. 1994). Overnight (24 h) cultures of DH5α, DH5α/pUC19 and DH5α/pFab were prepared in LB, LBA and LBT, respectively. Equal volumes of DH5α and plasmid bearing cultures were mixed, simultaneously plated onto selective and non-selective media and diluted 1:100 in 10 ml fresh LB. The mixed culture was incubated for 24 h with shaking at 37° C., followed by plating as above then diluted 1:100 in 10 ml of fresh LB. This procedure was repeated 5 times over six days. The numbers of ampicillin or triclosan resistant colonies were scored relative to the total CFUs.

Triclosan Selection of pFab Clones

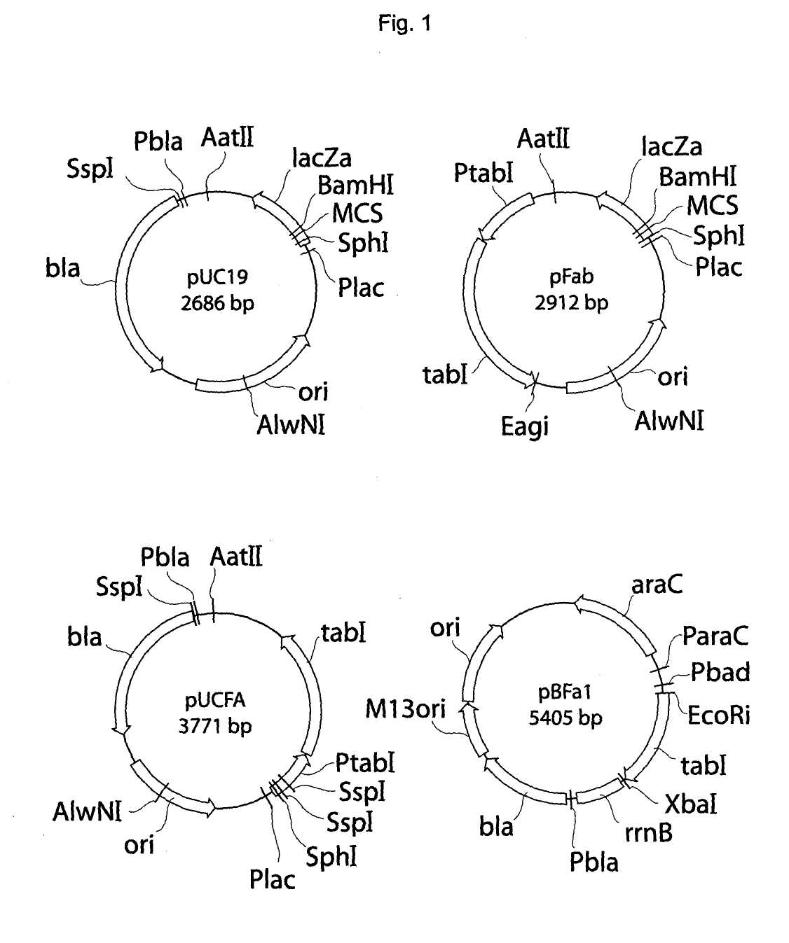

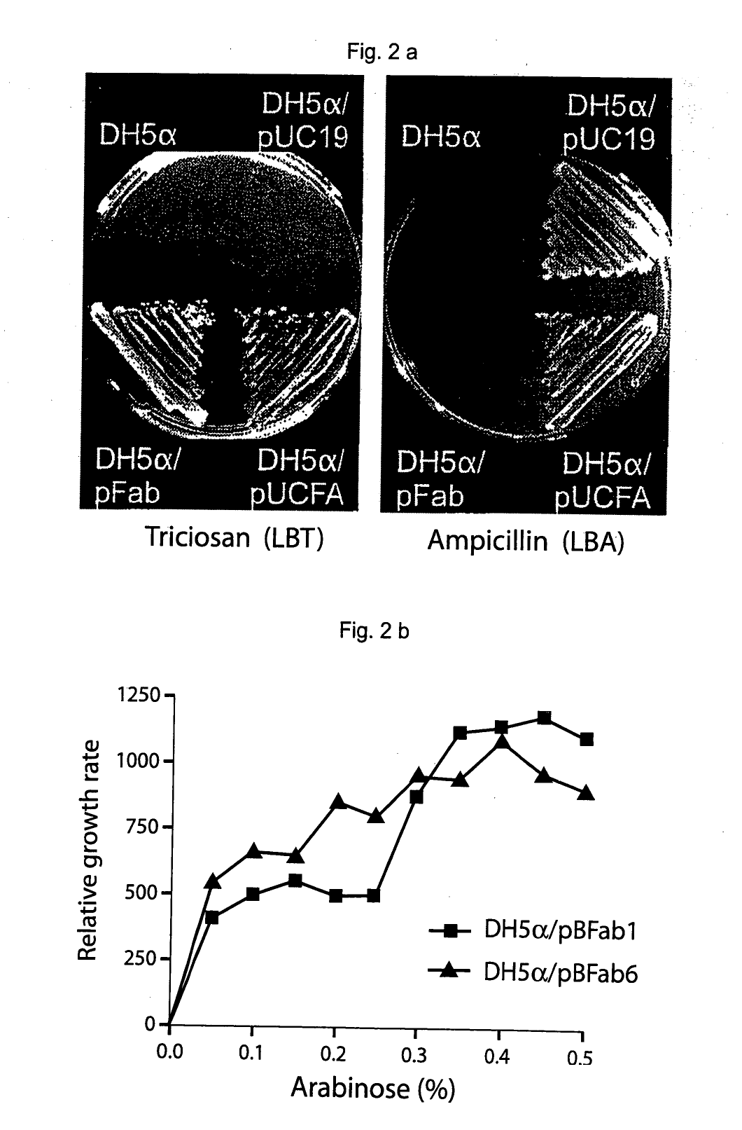

To test the potential of fabI as a selection marker for cloning, two vectors derived from pUC19 were constructed. First, we constructed pFab, where fabI together with its native promoter replaces bla in pUC19 for non-antibiotic selection using triclosan. Second, to enable selection with triclosan or ampicillin, we constructed pUCFA which contains the same fabI cassette cloned into the pUC19 MCS (FIG. 1). E. coli strain DH5α transformed with of pFab, pUCFA and pUC19 were selected on LB with triclosan (LBT) and LB with ampicillin (LBA). Colonies formed on LBT were more variable in size than colonies on LBA. However, triclosan-resistant colonies of all sizes maintained resistance and displayed uniform colony morphologies upon re-plating (FIG. 2a), and we did not observe plasmid-free resistant strains or satellite colonies.

To confirm that fabI expression mediates triclosan resistance, expression of fabI was placed under the control of the P_{BAD} promoter in pBAD18s (pBFab1) and pBAD18 (pBFab6) (Guzman et al. 1995). pBFab1 and pBFab6 clones were tested for resistance with arabinose induction. In the absence of arabinose, no growth was observed in LBT, but growth rates increased with increasing arabinose concentrations up to 0.4% (FIG. 2b). Therefore, pFab resistance to triclosan is due to expression of fabI.

Characterisation of pFab

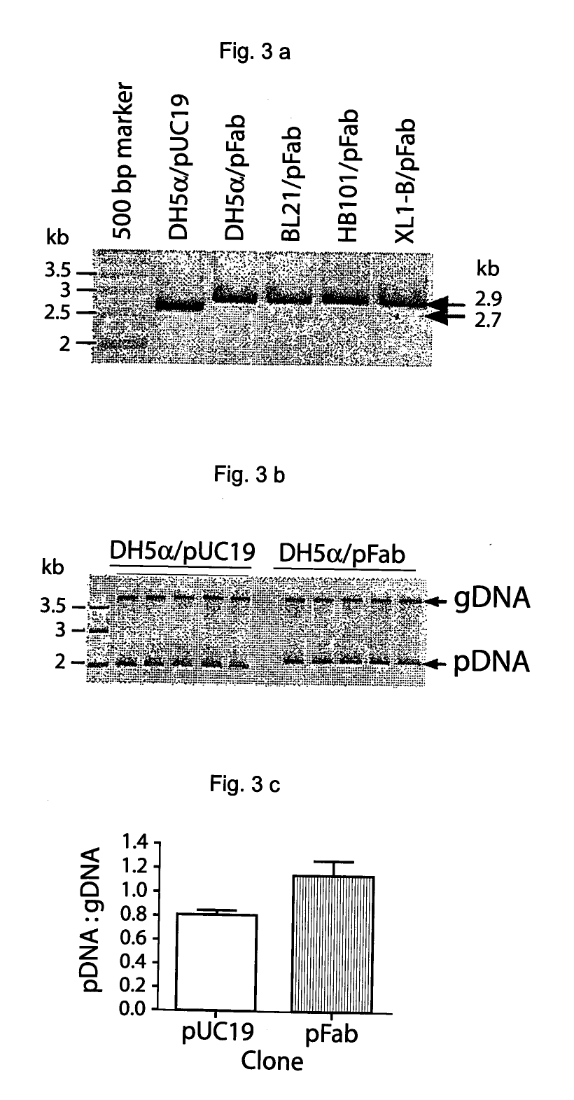

After selection of pFab transformants with triclosan, we then characterized the general properties pFab as a cloning vector, with pUC19 included for comparison (Table 1). The yield of pFab was 43% greater than pUC19 (Table 1) in DH5α transformants, and pFab preparations from three other commonly used E. coli cloning strains appeared to be similar in yield and plasmid integrity (FIG. 3a). Also, the copy number of pFab was 38% above that of pUC19 as measured by qPCR. This result was supported by isolating total DNA and then comparing the amounts of plasmid relative to genomic DNA in an agarose gel. The results indicate a 40% increase in plasmid DNA abundance in cells (FIG. 3b & c). Therefore, while not anticipated, pFab raises the copy number and plasmid yield relative to its parent pUC19.

To determine plasmid stability, we first scored triclosan resistant colony forming units relative to total colony forming units for DH5α/pFab. Surprisingly, we observed more colonies on LBT than LB, and we then considered the possibility that DH5α/pFab viability was somehow dependent on triclosan. To evaluate this possibility further, we determined plasmid stability by scoring α-complementation and found that pFab and pUC19 were stably maintained under selection, but that pFab was less stable than pUC19 without selection (Table 1). Therefore, while pFab clearly confers triclosan resistance, it also confers some form of requirement for the biocide triclosan.

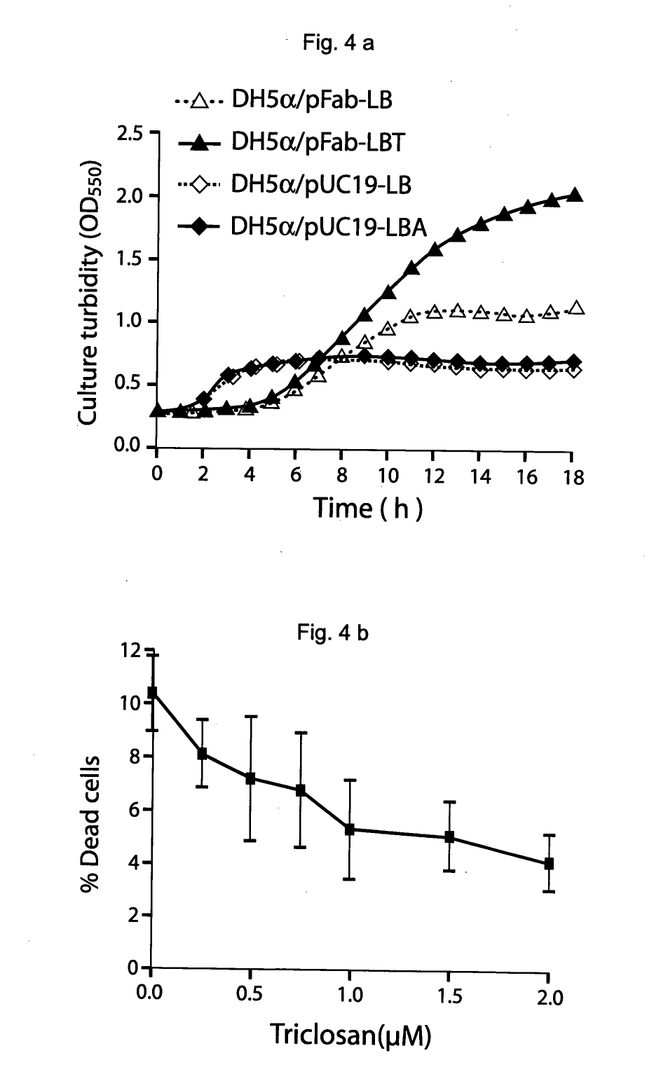

To investigate whether pFab carriage affects cell fitness, we examined the growth curve of DH5α/pFab with and without selection. We observed an overall longer lag phase and a higher final optical density, compared to DH5α/pUC19 (FIG. 4a). The final optical density of DH5α/pFab was greater in the presence of triclosan than in the absence. When we compared the growth rates (calculated from the exponential phase) of DH5α/pFab to DH5α/pUC19 either with or without selection, we found that DH5α/pFab grew faster than DH5α/pUC19 with selection, while the reverse was observed without selection (Table 1). Consistent with the results for plasmid stability, the growth rate data showed that pFab decreased fitness and the effect was suppressed by triclosan.

Effects of fabI Over-Expression and Triclosan on Host Fitness and Survival

As described above, we unexpectedly observed that cells containing pFab suffered from toxicity (cell death) and reduced fitness (growth rate or competition in mixed culture) in the absence of triclosan. In other words, pFab clones were dependent on triclosan for growth, despite triclosan being a potent biocide. A possible explanation is that excess FabI is toxic to cells, and FabI inhibition by triclosan suppresses this toxicity. To study these effects further, we assayed cell viability and death by using the viability stain SYTOX Green. Fluorescence microscopy of the SYTOX stained cells revealed elongated and dead cells in the absence of triclosan, and elongated and viable cells in 1 μM triclosan (data not shown). Also, the live and dead cell populations in DH5α/pFab culture were quantified by flow cytometry of SYTOX stained cells. The proportion of green fluorescent cells, or dead cells, decreased with increasing triclosan concentrations (FIG. 4b). Therefore, pFab is toxic and the growth effects can be suppressed by triclosan.

To investigate the fitness burden imposed by pFab and the suppression of this toxicity by triclosan, we monitored DH5α/pFab culture turbidity in media containing 0-2 μM triclosan and determined growth rate. As pFab was unstable without triclosan, DH5α/pUCFA grown in LBA was included in this experiment as a control to prevent competing plasmid-free cells. Growth rates of pUCFA and pFab clones were lowest without triclosan and highest at 1 μM triclosan, indicating a triclosan-dependent fitness effect in cells that carry pFab (FIG. 4c).

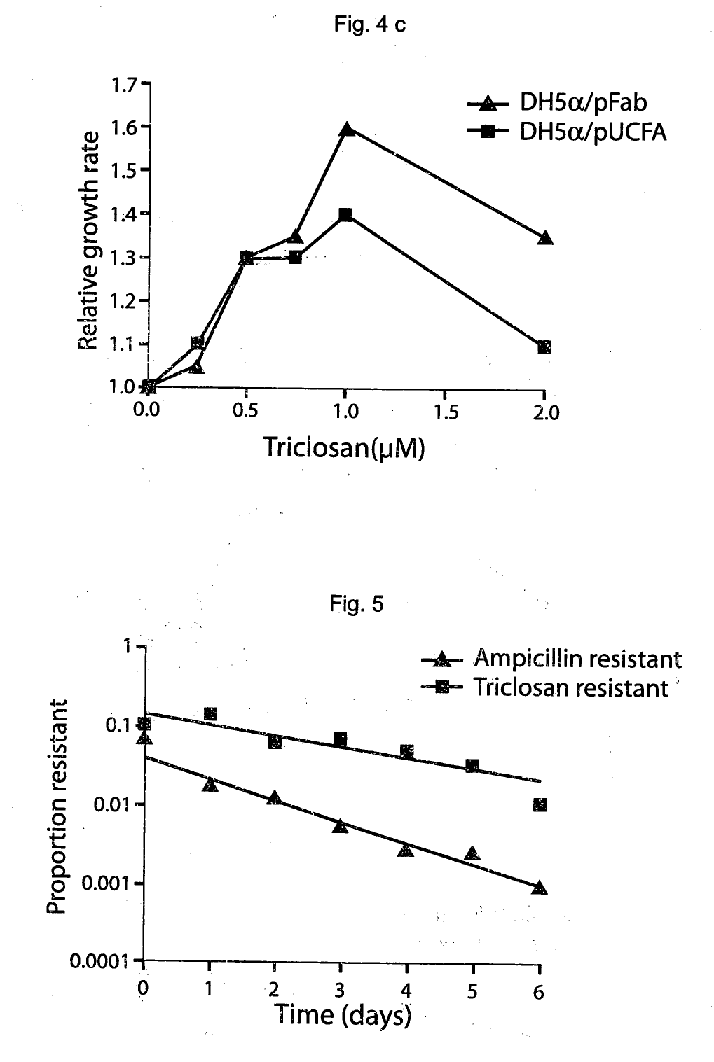

To address the question of whether triclosan resistance would persist outside of its intended application, we assessed the fitness of pUC19 and pFab clones in mixed culture with the plasmid-free parent strain, in the absence of selection. A growth competition assay showed that pFab persistence, and hence triclosan resistance, was weaker than that of pUC19 after co-culture for six days (FIG. 5) in the absence of triclosan. In this regard, the rate of plasmid loss for pFab was greater than pUC19, indicating that pFab is unstable or less competitive than pUC19 in the presence of wild type cells.

TABLE 1
Properties of pFab and pUC19

| Parameters^{a} | pUC19 | pFab |
|---|---|---|
| Transformation | 5.5 × 10⁷ ± 1.2 × 10⁷ | 2.2 × 10⁷ ± 3.6 × 10⁶ |
| efficiency^{b} (CFU/μg) | | |
| Plasmid yield^{c} (μg/ml) | 30 ± 2.1 | 42.9 ± 3.5 |
| Copy number^{d} | 141 ± 25 | 200 ± 33 |
| Stability (%) | | |
| With selection^{e} | 85.3 ± 10.8 | 99.5 ± 1.5 |
| Without selection^{f} | 56.7 ± 17.7 | 54.5 ± 14.9 |
| Relative growth rate^{g} | | |
| (ΔOD/Δt) | | |
| With selection | 1 | 1.2 ± 0.07 |
| Without selection | 1.1 ± 0.05 | 0.6 ± 0.02 |

^{a}Determined from five replicate cultures.
^{b}In chemically competent DH5α cells.
^{c}Plasmid yield from 1 ml of 18 h culture, determined by OD₂₆₀ absorbance.
^{d}Determined from the copy ratio of lacZα to dxs by qPCR.
^{e}Percentage of plasmid-bearing cells at 48 h in cultures grown with selection.
^{f}Percentage of plasmid-bearing cells at 48 h in cultures grown without selection.
^{g}Change in OD₅₅₀ over time of DH5α/pUC19 cultures in LB and DH5α/pFab in LB or LBT, relative to control DH5α/pUC19 cultures in LBA.

### CITATIONS

Bergler, H, P Wallner, A Ebeling, B Leitinger, S Fuchsbichler, H Aschauer, G Kollenz, G Högenauer, and F Turnowsky. 1994. “Protein EnvM is the NADH-dependent enoyl-ACP reductase (FabI) of Escherichia coli.” The Journal of biological chemistry 269 (8):5493-6.
Berti, T and S Caffau. 1963. Cross antibiotic-dependence in staphylococcus aureus. Bollettino chimico farmaceutico 102:868-71.
Cranenburgh, R M, J A Hanak, S G Williams, and D J. Sherratt. 2001. “Escherichia coli strains that allow antibiotic-free plasmid selection and maintenance by repressor titration.” Nucleic acids research 29 (5):E26.
Dabbs, E R and K Looman. 1981. “An antibiotic dependent conditional lethal mutant with a lesion affecting transcription and translation.” Molecular & general genetics: MGG 184 (2):224-9.
Dryselius, R, N Nekhotiaeva, and L Good. 2005. “Antimicrobial synergy between mRNA- and protein-level inhibitors.” The Journal of antimicrobial chemotherapy 56 (1):97-103.
Fraimow, H S, D L Jungkind, D W Lander, D R Delso, and J L Dean. 1994. “Urinary tract infection with an Enterococcus faecalis isolate that requires vancomycin for growth.” Annals of internal medicine 121 (1):22-6.
Goldstein, F, J Perutka, A Cuirolo, K Plata, D Faccone, J Morris, A Sournia, M D Kitzis, A Ly, G Archer, and A E Rosato. 2007. “Identification and phenotypic characterization of a {beta}-lactam-dependent, methicillin-resistant Staphylococcus aureus (MRSA).” Antimicrob Agents Chemother.
González, A, G Dávila, and E Calva. 1985. “Cloning of a DNA sequence that complements glutamine auxotrophy in Saccharomyces cerevisiae.” Gene 36 (1-2):123-9.
Guzman, L M, D Belin, M J Carson, and J. Beckwith. 1995. “Tight regulation, modulation, and high-level expression by vectors containing the arabinose PBAD promoter.” Journal of bacteriology 177 (14):4121-30.
Heath, R J, J R Rubin, D R Holland, E Zhang, M E Snow, and C O Rock. 1999. “Mechanism of triclosan inhibition of bacterial fatty acid synthesis.” The Journal of biological chemistry 274 (16):11110-4.
Heath, R J, Y T Yu, M A Shapiro, E Olson, and C O Rock. 1998. “Broad spectrum antimicrobial biocides target the FabI component of fatty acid synthesis.” The Journal of biological chemistry 273 (46):30316-20.
Kerasheva, S I. 1966. “[Participation of antibiotic-dependent strains of staphylococci in the pathological process].” Antibiotiki 11 (10):933-5.
Lee, C, J K, S G Shin, and S Hwang. 2006. “Absolute and relative QPCR quantification of plasmid copy number in Escherichia coli.” Journal of biotechnology 123 (3):273-80.
Lenski, R E, S C Simpson, and T T Nguyen. 1994. “Genetic analysis of a plasmid-encoded, host genotype-specific enhancement of bacterial fitness.” Journal of bacteriology 176 (11):3140-7.
Li, C P and A R. Rendina. 2006. U.S. Pat. No. 7,141,721-Enoyl-ACP reductases. Retrieved Jul. 8, 2007 (http://patft.uspto.gov/netacgi/nph-Parser?Sect1=PTO1&Sect2=HITOFF&d=PALL&p=1&u=%2Fnetahtm1%2FPTO %2Fsrc hnum.htm&r=1&f=G&1=50&s1=7141721.PN.&OS=PN/7141721&RS=PN/7141721)
Livak, K J and T D Schmittgen. 2001. “Analysis of relative gene expression data using real-time quantitative PCR and the 2(-Delta Delta C(T)) Method.” Methods (San Diego, Calif.) 25 (4):402-8.
Panagakos, Fotinos S, Anthony R Volpe, Margaret E Petrone, William DeVizio, Robin M Davies, and Howard M Proskin. 2005. “Advanced oral antibacterial/anti-inflammatory technology: A comprehensive review of the clinical benefits of a triclosan/copolymer/fluoride dentifrice.” The Journal of clinical dentistry 16 Suppl:S1-19.
Paterson, David L. 2006. “Resistance in gram-negative bacteria: Enterobacteriaceae.” American journal of infection control 34 (5 Suppl 1):S20-8; discussion S64-73.
Roth, B L, M Poot, S T Yue, and P J. Millard. 1997. “Bacterial viability and antibiotic susceptibility testing with SYTOX green nucleic acid stain.” Applied and environmental microbiology 63 (6):2421-31.
Sehgal, V, V S Shetty, S Mogra, G Bhat, M Eipe, S Jacob, and L Prabu. 2007. “Evaluation of antimicrobial and physical properties of orthodontic composite resin modified by addition of antimicrobial agents—an in-vitro study.” American journal of orthodontics and dentofacial orthopedics: official publication of the American Association of Orthodontists, its constituent societies, and the American Board of Orthodontics 131 (4):525-9.
Stokes, N R, J Sievers, S Barker, J M Bennett, D R Brown, I Collins, V M Errington, D Foulger, M Hall, R Halsey, H Johnson, V Rose, H B Thomaides, D J Haydon, L G Czaplewski, and J. Errington. 2005. “Novel inhibitors of bacterial cytokinesis identified by a cell-based antibiotic screening assay.” The Journal of biological chemistry 280 (48):39709-15.
Stuitj, R and J. Nijkamp. Selection of genetically manipulated organisms containing a target DNA sequence comprises using an enoyl-ACP reductase gene as a selectable marker.
Tennhammar-Ekman, B, L Sundström, and O Sköld. 1986. “New observations regarding evolution of trimethoprim resistance.” The Journal of antimicrobial chemotherapy 18 Suppl C:67-76.
Ward, W H, G A Holdgate, S Rowsell, E G McLean, R A Pauptit, E Clayton, W W Nichols, J G Colls, C A Minshull, D A Jude, A Mistry, D Timms, R Camble, N J Hales, C J Britton, and I W Taylor. 1999. “Kinetic and structural characteristics of the inhibition of enoyl (acyl carrier protein) reductase by triclosan.” Biochemistry 38 (38):12514-25.
Weitao, T, K Nordström, and S Dasgupta. 1999. “Mutual suppression of mukB and seqA phenotypes might arise from their opposing influences on the Escherichia coli nucleoid structure.” Molecular microbiology 34 (1):157-68.
Williams, S G, R M Cranenburgh, A M Weiss, C J Wrighton, D J Sherratt, and J A Hanak. 1998. “Repressor titration: a novel system for selection and stable maintenance of recombinant plasmids.” Nucleic acids research 26 (9):2120-4.
Winstanley, T G and R C Spencer. 1987. “Antibiotic dependence in a strain of Neisseria pharyngis.” The Journal of hospital infection 10 (1):87-90.
Wohlrab, J, G Jost, and D Abeck. 2007. “Antiseptic efficacy of a low-dosed topical triclosan/chlorhexidine combination therapy in atopic dermatitis.” Skin pharmacology and physiology 20 (2):71-6.
Xu, H H, L Real, and M W Bailey. 2006. “An array of Escherichia coli clones over-expressing essential proteins: a new strategy of identifying cellular targets of potent antibacterial compounds.” Biochemical and biophysical research communications 349 (4):1250-7.
Yue, T F, P G Dayton, and A B Gutman. 1963. Mutual suppression of the uricosuric effects of sulfinpyrazone and salicylate: a study in interactions between drugs. The Journal of clinical investigation 42:1330-9.

1.-27. (canceled)

28. A plasmid vector that expresses a growth essential gene at a level that makes growth of the host organism dependent on an inhibitor of the expressed gene.

29. A plasmid vector according to claim 28 wherein the growth essential gene is the gene of enoyl-ACP reductase (fabI) or its homologues, where vector-mediated expression of the said gene reduces the growth or survival of a host organism by being capable of expressing toxic levels of fabI, optionally where the open reading frame of the fabI gene is fused to an endogenous or heterologous gene sequence.

30. A plasmid vector according to claim 28 or 29 derived from pUC-derived plasmids, and/or wherein the plasmid vector is derived from pUC-derived plasmids having bla replaced with the fabI gene.

31. A host organism transformed with a plasmid vector according to any of claims 28 to 30, said organism being selected among bacteria, fungi and plant.

32. A method of enhancing the growth or survival of an organism according to claim 31 characterized by cultivating the organism that expresses from a plasmid a toxic level of an essential gene in the presence of an inhibiting amount of an antibacterial compound that targets the over-expressed essential gene product, whereby the toxic effects from the plasmid vector are suppressed by said inhibitor and the toxic effects of the inhibitor are suppressed by said plasmid vector in a mutually suppressive combination.

33. A method enhancing the growth or survival of an organism according to claim 32, wherein the gene is the fabI gene, optionally wherein said inhibitor is triclosan or a functional analogue of triclosan, optionally wherein triclosan is present in doses ranging from 100 nM to 10 μM during the cultivation.

34. A method according to claim 32 or 33, providing increased cell growth rates or yield of bacteria in a fermentation culture, and/or providing an increased recombinant protein expression, and/or providing an increased plasmid yield, and/or providing an increased metabolite production, and/or providing an increased bio-remediation activity of microorganisms, and/or providing enhanced vector stability, and/or providing an improved cell lysis efficiency, and/or providing a limited dissemination or spread of plasmid vector sequences.

35. A method producing by fermentation small molecule inhibitors capable of inhibiting growth essential genes using microorganisms that carry a vector according to any of claims 28 to 30, comprising the steps of culturing organisms according to claim 31 in such a manner that they produce as a metabolite a molecule that suppresses the toxic effects of the over-expressed gene.

36. A method of identifying or screening for small molecule inhibitors that bind and inhibit growth essential genes by culturing organisms that carry a vector according to any of claims 28 to 30 comprising the steps of subjecting selected inhibitor candidates to cultures and identifying molecules that are capable of suppressing the toxic effects of the growth essential gene.

37. A plasmid DNA vaccine product lacking an antibiotic resistance marker produced according to a method according to any of claims 32 to 34.

38. A recombinant whole cell vaccine product lacking an antibiotic resistance marker and is produced according to a method according to any of claims 32 to 34.

39. A method of producing plasmids while obtaining improvements in plasmid copy number, yield and stability, comprising:
transforming an organism with a plasmid vector according to any of claims 28 to 30 capable of expressing the gene of enoyl-ACP reductase (fabI);
culturing the organism under conditions of mutual suppression generated by the capacity of expressing toxic levels of enoyl-ACP reductase and the presence of a toxic, inhibitory amount of triclosan or a functional analogue of triclosan; and collecting the plasmids for further processing.

40. A method according to claim 39, wherein triclosan is present in an amount of 100 nM to 10 μM, preferably 0.5 to 2 μM, and more preferably in an amount about 1 μM.

41. A method according to claim 39 or 40, wherein the processing of plasmids includes inserting a heterologous or endogenous gene sequence, optionally wherein the heterologous or endogenous gene sequence is fused to the fabI gene.

42. A method according to any of claims 39 to 41, wherein the organism is E. coli.
